(19) United States
(12) Patent Application Publication
(10) Pub. No.: US 2015/0322463 A1
(43) Pub. Date: Nov. 12, 2015

(54) METHOD FOR OBTAINING BIOFUEL FROM LIGNOCELLULOSIC AND/OR AMYLACEOUS BIOMASS

(71) Applicant: BRAERG - GROUPO BRASILEIRO DE PESQUISAS ESPECIALIZADAS LTDA, Sorocaba (BR)

(72) Inventors: Douglas Spalato MARQUES, Tatui (BR); Marcos Spalato MARQUES, Sorocaba (BR)

(21) Appl. No.: 14/650,636

(22) PCT Filed: Dec. 3, 2013
(86) PCT No.: PCT/BR2013/000537

§ 371 Date: Jun. 9, 2015

(30) Foreign Application Priority Data
Dec. 13, 2012 (BR) BR 102012031841 5

(51) Int. Cl. C12P 7/10; C10L 1/02

(52) U.S. Cl. CPC C12P 7/10; C10L 1/02; C10L 2200/0469; C10L 2290/26; C10L 2290/544

(57) ABSTRACT

A method for obtaining biofuel from lignocellulosic and/or amylaceous biomass through fermentation in at least one step by using a consortium of microorganisms in order to obtain sugars that later will be converted into alcohols by using environmentally friendly and economically viable methods, as opposed to conventional acid and enzymatic hydrolysis.

### FIELD OF THE INVENTION

This invention refers to a method for obtaining biofuel from lignocellulosic and or amylaceous biomass through fermentation in at least one step by using a consortium of microorganisms in order to obtain sugars that later will be converted into alcohols by using environmentally friendly and economically viable methods, as opposed to conventional acid and enzymatic hydrolysis.

### BACKGROUND OF THE INVENTION

Nowadays the importance of biofuels is increasingly recognized. They are renewable sources of energy derived from organic matter and release a significantly smaller amount of pollutants into the atmosphere than such fossil fuels as oil derivatives.

Currently, sugarcane ethanol is produced by the alcoholic fermentation of sucrose—first-generation ethanol. Thus, cellulosic ethanol produced from plant cell wall polysaccharides is called second-generation ethanol. However, the step of chemical hydrolysis of the cell wall, which uses acid or basic solvents to loosen and break down plant cell wall polymers, releasing fermentable mono- and oligosaccharides is necessary for the production of cellulosic ethanol. However, in addition to the cost of chemical products used, collateral chemical residues may be produced. (Marcos S. Buckeridge, Marco S. Santos, Wanderley D. dos Souza, Amanda P. The paths for cellulosic ethanol in Brazil. USP. 2012).

Over the years, several methods of hydrolysis of cellulosic material have been proposed. The characteristic common to all of them is that the material must be first mechanically crushed in order to reduce the size of the particles and increase the surface of the material in contact with the medium.

Hydrolysis is usually carried out in two ways. In case of the acid hydrolysis, the use of large quantities of acid makes the medium habitable for any microorganism that ferments sugars resulting from the acid hydrolysis to ethanol, which makes the medium recovery step necessary so that it could be acid-free. This is one of the biggest disadvantages of this method because the recovery is a process that requires a large amount of energy, making this step very costly. Besides, the corrosive nature of the acid requires the use of high-cost alloys in pipes and heat exchangers.

In case of the enzymatic hydrolysis of cellulose to ethanol, there are pretreatment procedures intended to remove lignin and expose cellulose and hemicellulose to the action of the cellulase enzyme. However, the use of enzymes capable of degrading cellulose (cellulase) is still not viable because of its high cost (Nguyen, Q. A.; Sadler, J. N. (1991) Biores. Technol., 35, 275-282), low productivity, environmental risks involved, and logistics resources necessary to transport the enzymes to the biomass processing site.

The prior art discloses methods for producing ethanol which basically comprise two pathways: acid hydrolysis and enzymatic hydrolysis.

CN101544990 discloses a method for producing fuel by using lignocellulosic biomass that undergoes fermentation by inoculation of a cellulase-producing microorganism.

BRP10706009 discloses an alcoholic fermentation process that uses flocculent yeast strains in bioreactors, using vegetal biomass that contains sucrose, glucose, and fructose.

US2006177917 discloses a method for producing cellulolytic and/or hemicellulolytic enzymes by using residues from ethanol fermentation of hydrolysates and integrating the process for the production of second-generation ethanol, which contains such steps as a physical and chemical pretreatment, enzymatic hydrolysis, hydrolysate fermentation by using microorganisms, and alcohol separation and purification.

Although there is technical literature on methods for obtaining ethanol by fermenting glucose, the challenge is to obtain fermentable sugars from biomass by using environmentally friendly and economically viable methods that use microorganisms (fungi and bacteria) to remove cell walls.

Therefore, the purpose of this invention is a method for obtaining biofuel from lignocellulosic and or amylaceous biomass through fermentation in at least one step by using a consortium of microorganisms capable of producing enzymes under conditions necessary for degradation of the biomass, thus eliminating the biomass pretreatment steps and the use of isolated enzymes, and ensuring the obtainment of sugars that later will be converted into alcohols.

### SUMMARY

In one aspect there is provided a method for obtaining biofuel from lignocellulosic biomass, comprising at least one fermentation step for breaking down lignin, cellulose, hemicelluloses, starch, and converting sugars into alcohols and acids.

In another aspect there is provided a process for obtaining biofuel from lignocellulosic biomass by using microorganisms.

In a further aspect there is provided a process for obtaining biofuel from lignocellulosic biomass, eliminating the biomass pretreatment steps by using acids, alkalis, and isolated enzymes.

In yet another aspect there is provided a method for obtaining biofuel from lignocellulosic biomass by fully using all raw materials, thus providing an environmentally friendly and economically viable process.

In yet another aspect there is provided a method for obtaining biofuel from lignocellulosic biomass, providing around 30% w/v of 96% ethanol, as opposed to conventional methods for obtaining 8% w/v on the average.

### DETAILED DESCRIPTION OF THE INVENTION

For the purposes of this invention, the expression “modulation of enzyme production” shall mean the capacity of an organism to produce enzymes (increase, reduction or alteration) in accordance with the medium it is in.

The method for obtaining biofuel by using lignocellulosic and/or amylaceous biomass, subject matter of this invention, comprises a first step of particle size reduction of biomass such as sugarcane and banana crop residues, among others, in order to increase the surface area of contact.

Optionally, the fractionated lignocellulosic and/or amylaceous biomass goes on to the step of sterilization.

The sterilization of lignocellulosic biomass inhibits development of pollutants. The contamination increases the risk of inhibiting the yeast Saccharomyces and other microorganisms that may be used in this process through substrate competition and release of metabolites, which may lead to reduction in yield and productivity (Naves, Raquel Ferreira, Fernandes, Fernanda de Souza; Pinto, Osvaldo Gomes and Naves, Plínio Láazaro Faleiro. Microbial contamination in the processing steps and its influence on the fermentation yield at alcohol plants).

For mash fermentation, one or more microorganisms capable of producing and modulating production of enzymes that degrade lignin, cellulose, and hemicelluloses are preferably used, maintaining the temperature between 10° C. and 80° C., and pH between 2.0 and 12.0.

After lignin, cellulose, and hemicelluloses have been degraded, sugars and proteins dispersed in the mash are obtained, thus increasing the amounts of soluble solids in the mash. In this procedure, such species of microorganisms as Phanerochaete ssp., Gloeophylum ssp., Phellinus ssp., Coriolopsis ssp., Clostrodium ssp., Armillaria ssp., Chaetomium ssp., Serpulaceae ssp., Fibroporia ssp., Coniophora ssp., Aspergillus ssp. or Trichoderma ssp. are preferably used.

After achieving the desired concentration of reducing sugars, the mach may go through the step of sterilization to eliminate pollutants.

Then the mash continues to ferment using one or more amylolytic microorganisms, preferably those of the Baccilus spp. genus, maintaining the temperature between 10° C. and 50° C., and pH between 2.0 and 12.0 so that alpha-amylase enzymes catalyze the hydrolysis of glycosidic linkages.

Optionally, between 1.0 and 10.0% w/v of the ammonium salt composition is added.

Optionally, after achieving the desired concentration of reducing sugars, the mash may go on to the step of sterilization.

The mash continues to ferment using between 2.0 and 10.0% of one or more microorganisms of the Saccharomyces spp. genus, maintaining the temperature between 10° C. and 60° C., and pH between 2.0 and 12.0, converting the sugars present in the mush into alcohols, preferably, ethanol.

After achieving the desired concentration of reducing sugars, the mash goes through the alcohol extraction process.

Optionally, the amylolytic and/or cellulolytic and hemicellulolytic microorganisms may be added to the mash together with one or more microorganisms of the Saccharomyces ssp. Genus for one-step fermentation.

Citric, acetic, and lactic acids are produced during the fermentation process due to the amount of time in contact with the microorganism in the mash.

### EXPERIMENT

The lignocellulosic material from sugarcane waste (bagasse and straw), and mashed banana fruit, stem, pseudostem, and leaves without any kind of pretreatment were used in this experiment.

Microorganisms were selected to degrade the lignocellulosic material into fermentable sugars.

TABLE 1
Quantity of fermentable sugars

| | Fermentation | | |
|---|---|---|---|
| | time | Degrees Brix | pH |
| Addition of | 1^{st} day | 4 | 4.5 |
| microorganism 1 | 2^{nd} day | 2 | 4.2 |
| | 3^{rd} day | 19 | 3.8 |
| Addition of | 4^{th} day | 10 | 4.0 |
| microorganism 2 | 5^{th} day | 24 | 4.2 |
| Addition of | 6^{th} day | 12 | 4.3 |
| microorganism 3 | 7^{th} day | 1 | 4.5 |

As shown in the Table 1, there was a large-scale growth of the quantity of fermentable sugars (represented in degrees Brix) during the fermentation process, which means that the microorganisms were capable of modulating their enzymatic production in order to degrade the lignocellulosic material and produce sugars.

30% of 96° GL ethanol was obtained without producing toxic byproducts, which therefore means that it was a clean process.

1. Method for obtaining biofuel from lignocellulosic and/or amylaceous biomass comprising the steps of:
a) reduction of particles of the lignocellulosic and/or amylaceous biomass;
b) fermentation of the mash by using one or more microorganisms capable of producing and modulating production of enzymes that degrade lignin, cellulose, and hemicelluloses at temperatures between 10° C. and 80° C., and ph between 2.0 and 12.0, followed by fermentation by using one or more microorganisms, preferably those of the Baccilus ssp. genus, at temperatures between 10° C. and 50° C., and pH between 2.0 and 12.0, and between 2.0 and 10.0% of one or more microorganisms of the Saccharomyces spp. genus at temperatures between 10° C. and 60° C. and, pH between 2.0 and 12.0;
c) extraction of alcohols.

2. Method for obtaining biofuel from lignocellulosic and/or amylaceous biomass according to claim 1 wherein, optionally, said fractionated lignocellulosic or amylaceous biomass goes through the step of sterilization.

3. Method for obtaining biofuel from lignocellulosic and/or amylaceous biomass according to claim 1 wherein lignin, cellulose, and hemicelluloses are degraded by one or more microorganisms of the following genera: Phanerochaete ssp., Gloeophylum ssp., Phellinus ssp., Coriolopsis ssp., Clostrodium ssp., Armillaria ssp., Chaetomium ssp., Serpulaceae ssp., Fibroporia ssp., Coniophora ssp., Aspergillus ssp. or Trichoderma ssp.

4. Method for obtaining biofuel from lignocellulosic and/or amylaceous biomass according to claim 1 wherein said amylolytic microorganism is preferably of the Baccilus spp. genus.

5. Method for obtaining biofuel from lignocellulosic and/or amylaceous biomass according to claim 1 wherein between 1.0 and 10.0% w/v of the ammonium salt composition is preferably added during fermentation, using one or more amylolytic microorganisms.
